(19) United States
(12) Patent Application Publication
(10) Pub. No.: US 2023/0398090 A1
(43) Pub. Date: Dec. 14, 2023

(54) METHOD OF TREATING CORONAVIRUS INFECTION BY ADMINISTRATION OF ETHYL MERCURY OR THIOL DERIVATIVE THEREOF

(71) Applicant: BEECH TREE LABS, INC., Delanson, NY (US)

(72) Inventor: John McMichael, Delanson, NY (US)

(21) Appl. No.: 17/913,086

(22) PCT Filed: Mar. 24, 2021
(86) PCT No.: PCT/US21/23846

§ 371 Date: Sep. 20, 2022

Related U.S. Application Data
(60) Provisional application No. 63/001,086, filed on Mar. 27, 2020.

(51) Int. Cl. A61K 31/305; A61P 31/14

(52) U.S. Cl. CPC A61K 31/305; A61P 31/14

(57) ABSTRACT

Disclosed herein are methods of treating a coronavirus infection by administration of ethyl mercury or thiol derivative thereof in an amount effective to treat the coronavirus infection.

### CROSS-REFERENCE TO RELATED APPLICATION

This application is a national phase entry under 35 USC § 371 of International Application No. PCT/US2021/23846, filed Mar. 24, 2021, which claims the benefit of priority to U.S. Provisional Patent Application No. 63/001,086, filed Mar. 27, 2020, the entire disclosures of which are incorporated herein by reference in their entirety.

### BACKGROUND OF THE INVENTION

Severe acute respiratory syndrome-related coronavirus (SARS-CoV) is a member of the genus Betacoronavirus and subgenus Sarbecoronavirus and is a species of coronavirus that infects humans, bats and certain other mammals. It is an enveloped positive-sense single-stranded RNA virus that enters its host cell by binding to the ACE2 receptor. Two strains of the virus have caused outbreaks of severe respiratory diseases in humans: SARS-CoV (or SARS-CoV-1), which caused an outbreak of severe acute respiratory syndrome (SARS, or SARS-1) between 2002 and 2003, and SARS-CoV-2, which since late 2019 has caused a pandemic of coronavirus disease 2019 (COVID-19).

Influenza virus vaccines, and their components, have been used to treat other virus infections, such as herpes virus infections, as reported in Lieberman, Clinical Ecology, 7(3):51-54 (1990) and McMichael U.S. Pat. Nos. 4,521,405 and 4,880,626, all of which are incorporated herein by reference. Influenza vaccines induce a modified immune response such that symptoms were alleviated as a consequence of neutralizing the body's response to the infectious agent by stimulating suppressor T-cells. The T-cells in turn down-regulated effector cells and thus interrupted the allergic-type reaction induced by simultaneous lysis of many cells infected with the influenza virus. However, the results reported by McMichael and Lieberman attributed the anti-herpes virus effects of influenza virus vaccines to the presence of thimerosal, a preservative present in commercially available influenza virus vaccines, and not to a portion of the killed influenza virus, such as a surface antigen. Further, it has been discovered that the anti-herpes virus activity of influenza virus vaccines is attributable to the presence of thimerosal in the tested compositions and that herpes virus infections can be treated with thimerosal uncombined with or in the absence of influenza virus vaccine. Related to this is the disclosure of Manfuso, U.S. Pat. No. 4,803,991 which teaches the treatment of herpes virus infections by the topical administration of thimerosal, but does not teach or suggest the administration of thimerosal systemically.

U.S. Pat. No. 9,682,058 discloses the use of ethyl mercury or thiol derivative thereof for the treatment of an adenoviral infection, a human papilloma virus infection, a polyoma virus infection, and a pox virus infection.

There remains an urgent need for therapeutic antiviral treatment for coronavirus infections such as COVID 19.

### SUMMARY OF THE INVENTION

Described herein is a method of treating a coronavirus infection a subject comprising administering ethyl mercury or thiol derivative or salts thereof to the subject in an amount effective to treat the coronavirus infection. In some embodiments, the coronavirus infection is caused by SARS-CoV-1 or SARS-CoV-2.

In some embodiments, the thiol derivative of ethyl mercury is selected from the group consisting of an alkylthiol-ethyl mercury derivative and an arylthiol ethyl mercury derivative. In one embodiment, the thiol derivative of ethyl mercury is thimerosal. The term “thiol derivative of ethyl mercury” as used herein means a compound having a mercury sulfur bond and capable of releasing ethyl mercury or providing comparable therapeutic effects.

In some embodiments, the administration step comprises a route of administration selected from the group consisting of sublingual and subcutaneous administration.

In one embodiment, the thimerosal is administered in a dosage of about 0.05 μg to about 500 μg. In another embodiment, the thimerosal is administered in a dosage of about 0.05 μg to about 50 μg. In yet another embodiment, the thimerosal is administered in a dosage of about 0.2 μg. In another embodiment, thimerosal is administered sublingually as a drop (in a dose of 0.05 cc in a pharmaceutically acceptable carrier or excipient). In some embodiments, thimerosal is administered at least three times a day.

The subjects treated in the methods disclosed herein in its many embodiments are desirably human subjects, although it is to be understood that the principles of the presently disclosed subject matter indicate that the presently disclosed subject matter is effective with respect to invertebrate and to all vertebrate species, including mammals, which are intended to be included in the term “subject”. Moreover, a mammal is understood to include any mammalian species in which treatment or prevention of a viral infection described herein is desirable, particularly agricultural and domestic mammalian species.

While not intending to be bound by any specific theory of invention it is believed that thimerosal interferes with post-receptor translational signals (phosphorylation-protein synthesis complexes) to disrupt post-viral infected cellular tubulin synthesis and viral replication requiring the utilization of the host cell cascading protein synthesis mechanisms. Genomic studies indicate thimerosal down-regulates several pro-inflammatory cytokines while up-regulating other anti-inflammatory genes. These suggest that this novel treatment approach for COVID-19 viral infections may be capable of stopping intracellular viral replication and spread of viruses to adjacent cells resulting in decreased duration of the COVID-19 infection.

It is believed that interruption of intracellular viral replication by thimerosal may disrupt viral spread and infection of adjacent host cells, resulting in decreased viral load, shortened mean duration and severity of disease, and increased survival. Administration of thimerosal may alleviate symptoms of SARS-CoV-2 infection and reduce or eliminate viral shedding, thus preventing hospitalization of infected patients and facilitating treatment in an outpatient setting.

Treatment of COVID according to the present method results in improvements in the biometric measures for treated subjects including but not limited to oxygen saturation, temperature, level of pain, coughing, pulse, sore throat, “COVID toes” respiratory rate, sense of taste and sense of smell.

### DETAILED DESCRIPTION OF THE INVENTION

In one aspect, described herein is a method of treating a coronavirus infection in a subject comprising administering ethyl mercury or thiol derivative or salts thereof to the subject in an amount effective to treat the coronavirus infection. In some embodiments, the coronavirus infection is cause by SARS-CoV-1 or SARS-CoV-2.

In some embodiments, the thiol derivative of ethyl mercury is selected from the group consisting of an alkylthiol-ethyl mercury derivative and an arylthiol ethyl mercury derivative.

As used herein, the term “alkyl” refers to straight chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, ester, carboxylic acid, amide, guanidine, and amino.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

The salts, e.g., pharmaceutically acceptable salts, of the compounds of ethyl mercury or thiol derivatives thereof may be prepared by reacting the appropriate base or acid with a stoichiometric equivalent of the ethyl mercury or derivative thereof. Similarly, pharmaceutically acceptable derivatives (e.g., esters), metabolites, hydrates, solvates and prodrugs of ethyl mercury may be prepared by methods generally known to those skilled in the art. Thus, another embodiment provides compounds that are prodrugs of ethyl mercury. In general, a prodrug is a compound which is metabolized in vivo (e.g., by a metabolic transformation such as deamination, dealkylation, de-esterification, and the like) to provide an active compound. A “pharmaceutically acceptable prodrug” means a compound which is, within the scope of sound medical judgment, suitable for pharmaceutical use in a patient without undue toxicity, irritation, allergic response, and the like, and effective for the intended use, including a pharmaceutically acceptable ester as well as a zwitterionic form, where possible, of ethyl mercury. Examples of pharmaceutically-acceptable prodrug types are described in Higuchi and Stella, Pro-drugs as Novel Delivery Systems, Vol. 14 of the A.C.S. Symposium Series, and in Roche, ed., Bioreversible Carriers in Drug Design, American Pharmaceutical Association and Pergamon Press, 1987, both of which are incorporated herein by reference.

The compounds and compositions described herein may also include metabolites. As used herein, the term “metabolite” means a product of metabolism of a compound of the embodiments or a pharmaceutically acceptable salt, analog, or derivative thereof, that exhibits a similar activity in vitro or in vivo to ethyl mercury. The compounds and compositions described herein may also include hydrates and solvates. As used herein, the term “solvate” refers to a complex formed by a solute (herein, ethyl mercury) and a solvent. Such solvents for the purpose of the embodiments preferably should not negatively interfere with the biological activity of the solute. Solvents may be, by way of example, water, ethanol, or acetic acid. In view of the foregoing, reference herein to a particular compound or genus of compounds will be understood to include the various forms described above, including pharmaceutically acceptable salts, esters, prodrugs, metabolites and solvates thereof.

Appropriate dosages may be ascertained through the use of established assays for determining dose-response and toxicity and side-effect data. Typically, a pharmaceutical dosage unit for the delivery of ethyl mercury or thiol derivative thereof comprises a liquid or solid carrier and an effective amount of ethyl mercury or thiol derivative thereof to treat a viral infection described herein. One suitable carrier for sublingual administration comprises a phenylated saline solution. In one embodiment, the thiol derivative of ethyl mercury is thimerosal. Effective amounts of thimerosal range from about 0.05 μg to 500 μg thimerosal with about 0.1 μg to about 50 μg thimerosal being preferred and about 0.2 μg (200 ng) thimerosal being particularly preferred.

In some embodiments, thimerosal is administered 1, 2, 3, 4, 5, 6, 7, 8, 9, 10 or more times daily for a period of 1, 2, 3, 4, 5, 6 or more weeks. Additional therapy may be administered on a period basis, for example, daily, weekly or monthly.

According to a preferred embodiment of the invention subjects are treated by sublingual administration of a 200 ng dose of thimerosal in 0.05 ml drop of saline administered sublingually every 15 minutes for one hour followed by one dose per hour until bedtime. On days two and three of treatment one sublingual drop containing 200 ng of thimerosal is administered six times daily spaced throughout waking hours and therapy is continued on days 4 through 7 as needed by administration of one drop containing 200 ng of thimerosal as frequently as every 30 minutes.

In some embodiments, the ethyl mercury (or thiol derivative or salts thereof) is formulated in compositions that include at least one pharmaceutically acceptable carrier or excipient. Generally, this entails preparing compositions that are essentially free of pyrogens, as well as other impurities that could be harmful to humans or animals.

As used herein, “pharmaceutically acceptable carrier or excipient” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

In some embodiments, the administration of ethyl mercury (or thiol derivative or salts thereof) is carried out in a variety of conventional ways, including, but not limited to, oral ingestion, sublingual application, subcutaneous, intraperitoneal, and parenteral or intravenous injection. Sublingual administration to the subject is preferred. The treatment may consist of a single dose or a plurality of doses over a period of time.

For oral administration, ethyl mercury (or thiol derivatives or salts thereof) can be combined with pharmaceutically-acceptable carriers well known in the art. Such carriers enable the compound to be formulated as tablets, pills, dragees, capsules, liquids, gels, syrups, slurries, suspensions and the like, for oral ingestion by a subject to be treated. Pharmaceutical preparations for oral use can be obtained using a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures.

Formulations for parenteral administration include aqueous solutions of ethyl mercury (or thiol derivative or salts thereof). Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the ethyl mercury (or thiol derivative or salts thereof) may be in powder form for constitution with a suitable vehicle, e.g., sterile pyrogen free water, before use.

The compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The following examples are illustrative and are not intended to limit either the scope or spirit of the invention.

### EXAMPLE 1

According to this example a 29 year old woman presented with a positive coronavirus (SARS-CoV-2) diagnostic test, a fever of 104° F., slight shortness of breath and a cough for three days during which she was treated by administration of acetaminophen. She was then treated by sublingual administration of a 200 ng dose of thimerosal in 0.05 ml drop of saline administered sublingually every 15 minutes for one hour followed by one dose per hour until bedtime. All of the subject's symptoms then resolved 20-24 hours after initial treatment.

### EXAMPLE 2

A 34 year old male subject who had a spouse with similar symptoms presented with symptoms of coronavirus (SARS-CoV-2) infection including fever (103° F.), lethargy, shortness of breath and achiness which he had suffered for two to three days prior to treatment. Treatment was then initiated by sublingual administration of a 200 ng dose of thimerosal in 0.05 ml drop of saline administered sublingually every 15 minutes for one hour followed by one dose per hour until bedtime. All of the subject's symptoms then resolved within 24 hours after initial treatment.

### EXAMPLE 3

According to this example a 73 year old man presented with presumptive coronavirus (SARS-CoV-2) including a fever, weakness, shortness of breath and a cough. He was then treated by sublingual administration of a 200 ng dose of thimerosal in 0.05 ml drop of saline administered sublingually every 15 minutes for one hour followed by one dose per hour until bedtime. On days two and three of treatment he was treated with one sublingual drop containing 200 ng of thimerosal administered six times daily spaced throughout waking hours. All of the subject's symptoms then resolved 72 hours after initial treatment with the exception of weakness which only partially improved.

### EXAMPLE 4

According to this example an 18 year old woman presented after several days of symptoms with a positive coronavirus (SARS-CoV-2) diagnostic test, a cough, mild fever, aching, decreased energy and stomach pain. She was then treated by sublingual administration of a 200 ng dose of thimerosal in 0.05 ml drop of saline administered sublingually every 15 minutes for one hour followed by one dose per hour until bedtime and on day two was treated with one sublingual drop containing 200 ng of thimerosal administered six times daily spaced throughout waking hours. After less than 48 hours of therapy the subject reported diminution of all symptom, increased energy, increased appetite and improved ability to taste although her chest pain was not completely eliminated.

### EXAMPLE 5

According to this example a double-blind placebo-controlled study is conducte of superiority in subjects 40 years and older and with a positive laboratory diagnosis of SARS-CoV-2 infection.

A maximum of up to 60 subjects are enrolled into the study in order to ensure 40 completed subjects. Potential subjects are recruited from persons who have tested positively for COVID-19. Only subjects who contact the site to express interest within 36 hours of receiving a positive test result are considered for the trial. Subjects are be randomized to active treatment (thimerosal) or placebo at a 1:1 ratio.

The parallel group assignment are only be maintained until the completion of a minimum of 48 hours of blinded study drug dosing, at which point all subjects receive the active treatment/drug (Thimerosal). Although this will limit the as-randomized analyses to only the first 48-hours of the study, all subjects continue to be followed for 10 days to monitor adverse events as well as laboratory results. Forty subjects age 40 and older with a positive COVID-19 test (SARS-CoV-2 diagnostic test) are treated. Twenty (20) subjects are randomized to placebo and twenty (20) subjects to active treatment (BTL-TML-COVID) for 48 hours of dosing. Following a minimum of 48 hours of blinded dosing, all subjects receive active drug. Therapy comprises administration of sublingual administration of a 200 ng dose of thimerosal in 0.05 ml drop of saline administered sublingually every 15 minutes for one hour followed by one dose per hour until bedtime and on days two and three by sublingual administration of one 0.05 ml drop containing 200 ng of thimerosal administered six times daily spaced throughout waking hours. On days four through sixteen the subjects were treated by sublingual administration of one 0.05 ml drop containing 200 ng of thimerosal administered four times daily spaced throughout waking hours.

The subjects are evaluated by telemedicine visits for 17 days for the following biometric measures: oxygen saturation, temperature, level of pain, coughing, pulse, sore throat, “COVID toes” respiratory rate, sense of taste and sense of smell.

Primary Objective

The primary objective is to measure the mean severity of disease following administration of low-dose sub-lingual thimerosal oral solution using the Physical Component Summary of the SF-36 Quality of Life instrument.

Secondary Objective

The secondary objectives are to measure the incidence and severity of adverse events following administration of low-dose sub-lingual thimerosal oral solution and to measure the Mental Component Summary of the SF-36 as well as biometric measures of severity of symptoms associated with COVID-19.

Tertiary Objective

Tertiary objectives are to measure the duration of time over which SARS-CoV-2 diagnostic testing indicates the continued presence of the virus using nasal swabs. following administration of low-dose sub-lingual thimerosal oral solution.

The design is a double-blind placebo-controlled study of superiority in subjects 40 years and older and with a positive laboratory diagnosis of SARS-CoV-2 infection.

A maximum of up to 60 subjects are enrolled into the study in order to ensure 40 completed subjects. Potential subjects are recruited from persons who have tested positively for COVID-19. Only subjects who contact the site to express interest within 36 hours of receiving a positive test result will be considered for the trial. Subjects are randomized to active treatment with thimerosal or placebo at a 1:1 ratio.

The parallel group assignment are be maintained until the completion of a minimum of 48 hours of blinded study drug dosing, at which point all subjects receive the active treatment/drug (thimerosal). Although this will limit the as-randomized analyses to only the first 48-hours of the study, all subjects continue to be followed for 10 days to monitor adverse events as well as laboratory results.

Efficacy is measured by improvements in Quality of Life, as shown primarily through the Physical Component Summary of the SF-36. Differences between the treated and control groups are compared. Measures of Mental Component Summary of the SF-36 are likewise compared between the two groups, along with changes in the severity of symptoms associated with COVID-19.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed:

1. A method of treating a coronavirus infection in a subject comprising administering ethyl mercury or thiol derivative thereof to the subject in an amount effective to treat the viral infection.

2. The method of claim 1, wherein the coronavirus infection is caused by a member selected from the group consisting of SARS-CoV-1 and SARS-CoV-2.

3. The method of claim 2, wherein the coronavirus infection is caused by SARS-CoV-2.

4. The method of claim 1, wherein the thiol derivative of ethyl mercury is thimerosal.

5. The method of claim 1, wherein the subject is human.

6. The method of claim 1, wherein treatment results in improvement in one or more biometric measures selected from the group consisting of oxygen saturation, temperature, level of pain, coughing, pulse, sore throat, “COVID toes” respiratory rate, sense of taste and sense of smell.

7. The method of claim 1, wherein the administering step comprises a route of administration selected from the group consisting of sublingual and subcutaneous administration.

8. The method of claim 1, wherein the ethyl mercury or thiol derivative thereof is administered sublingually.

9. The method of claim 8, wherein the ethyl mercury or thiol derivative is administered at a dosage range of about 0.05 μg to about 500 μg.

10. The method of claim 8, wherein the ethyl mercury or thiol derivative is administered at a dosage range of about 0.2 μg to about 50 μg.

11. The method of claim 8, wherein the ethyl mercury or thiol derivative is administered at a dose of about 0.2 μg.

12. The method of claim 8, wherein the ethyl mercury or thiol derivative is administered in one drop intervals at least three times a day.
